(19) United States
(12) Patent Application Publication
(10) Pub. No.: US 2020/0180336 A1
(43) Pub. Date: Jun. 11, 2020

(54) THERMOSENSITIVE RECORDING MATERIAL AND LAMINATE

(71) Applicant: Mitsubishi Chemical Corporation, Chiyoda-ku (JP)

(72) Inventor: Keiichiro INADA, Chiyoda-ku (JP)

(73) Assignee: Mitsubishi Chemical Corporation, Chiyoda-ku (JP)

(21) Appl. No.: 16/784,733

(22) Filed: Feb. 7, 2020

Related U.S. Application Data
(63) Continuation of application No. PCT/JP2018/029662, filed on Aug. 7, 2018.

(30) Foreign Application Priority Data
Aug. 9, 2017 (JP) 2017-154558

(51) Int. Cl. B41M 5/333; B41M 5/323; C07C 309/75; B41M 5/42

(52) U.S. Cl. CPC B41M 5/3336; B41M 5/323; B41M 5/305; B41M 5/423; C07C 309/75

(57) ABSTRACT

An object is to provide a thermosensitive recording material and a laminate which are excellent in color development sensitivity and favorable in storage characteristics such as heat resistance, plasticizer resistance, moisture resistance, oil resistance, and grease resistance. A thermosensitive recording material in which a thermosensitive recording layer is provided on a support, wherein the thermosensitive recording layer contains a specific diphenylsulfone compound and an analog compound thereof at a specific ratio. A laminate in which a thermosensitive recording layer is provided on a support, wherein the thermosensitive recording layer contains a specific diphenylsulfone compound and an analog compound thereof at a specific ratio.

### CROSS-REFERENCE TO RELATED APPLICATION

This is a continuation of International Application PCT/JP2018/029662, filed on Aug. 7, 2018, and designated the U.S., and claims priority from Japanese Patent Application 2017-154558 which was filed on Aug. 9, 2017, the entire contents of which are incorporated herein by reference.

### TECHNICAL FIELD

The present invention relates to a thermosensitive recording material and a laminate which are favorable in color development sensitivity and which are favorable in heat resistance, moisture resistance, plasticizer resistance, oil resistance, and grease resistance.

### BACKGROUND ART

Thermosensitive recording materials having thermosensitive recording layers mainly containing color-developers which, when heated with leuco dyes (hereinafter, sometimes abbreviated as “dyes”), react therewith to thereby develop colors, are generally widely in practical use in applications of paper, films, cards, and the like. For example, thermal printers with built-in thermal heads are used for recording on such thermosensitive recording materials. Such thermosensitive recording systems are characterized by (a) not making any noise during recording, (b) requiring neither development, nor fixing, (c) being maintenance-free, (d) being relatively inexpensive in terms of equipment, (e) being compact, and (f) providing very clear color development, as compared with other recording systems which have been conventionally in practical use, and such systems are widely used in not only Point of sale (POS) labels for performing commodity management and recording at selling, like receipts, but also logistics/foods labels, cash vouchers, medical charts, and the like. In recent years, items have been scanned from barcodes or the like, and thermosensitive paper has been demanded which allows for a clear contrast between a print area and a blank area. On the other hand, such thermosensitive paper, which is to be touched with a human hand or to be in contact with any oil of foods, is thus demanded to have storage stability of a print area against oil and/or to have storage stability of a print area against hand cream (“grease resistance” is adopted as an index for evaluation in the present invention).

It is particularly important to select a color-developer forming a thermosensitive recording layer, as a factor having a large effect on color development sensitivity, storage stability of a print area and storage stability of a blank area, and various color-developers have been heretofore studied. For example, Patent Literature 1 describes 4-allyloxy-4′-hydroxydiphenylsulfone and Patent Literature 2 and 3 each describe 4-propoxy-4′-hydroxydiphenylsulfone, which is color-developer favorable in color development sensitivity and also favorable in storage stability of a blank area.

On the other hand, Patent Literature 4 describes a mixture of bis(4-allyloxydiphenyl)sulfone, 4,4′-dihydroxydiphenylsulfone and/or 2,4-dihydroxydiphenylsulfone with 4-allyloxy-4′-hydroxydiphenylsulfone, as a color-developer favorable in sensitivity and favorable in heat-resistant stability and wet-heat resistant stability of an image. Furthermore, Patent Literature 5 describes a mixture of alkylated bis(4-hydroxyphenyl)sulfone such as 4-hydroxyphenyl-3′-isopropyl-4′-hydroxyphenylsulfone with 4-isopropoxy-4′-hydroxydiphenylsulfone which is a color-developer, in order to prevent formation of any hydrate which causes background contamination in production and storage of a dispersion liquid.

### CITATION LIST

### Patent Literature

### Patent Literature 1: JP 2006-175673 A

### Patent Literature 2: WO 84/02882

### Patent Literature 3: WO 91/11433

### Patent Literature 4: JP 2006-44093 A

### Patent Literature 5: JP H8-324126 A

### SUMMARY OF INVENTION

### Technical Problem

According to studies of the present inventor, it has been found that the 4-allyloxy-4′-hydroxydiphenylsulfone described in Patent Literature 1, although it is favorable in color development sensitivity and storage stability of a blank area, is insufficient in function as a compound by itself for use as a color-developer because storage stability of a print area is inferior and thus a diphenylsulfone crosslinking type compound is used as a stabilizer for an improvement in the storage stability. It has also been found that the 4-propoxy-4′-hydroxydiphenylsulfone described in Patent Literature 2 and 3, although is favorable in color development sensitivity and storage stability of a blank area, has the problem of being insufficient in storage stability of a print area, for example, heat resistance, moisture resistance, plasticizer resistance, oil resistance, and grease resistance.

According to studies of the present inventor, it has been found that the mixture of a color-developer described in Patent Literature 4, although it is enhanced in storage characteristics such as heat resistance, moisture resistance and plasticizer resistance, it is not enhanced in grease resistance at all. Furthermore, the 4-isopropoxy-4′-hydroxydiphenylsulfone described in Patent Literature 5 is inferior in heat-resistant stability (heat-resistant ground color) of a blank area.

That is, there is not currently found any thermosensitive recording material which is excellent in color development sensitivity and which satisfies all heat resistance, moisture resistance, plasticizer resistance, oil resistance, and grease resistance, among color-developers conventionally developed, including the color-developers of Patent Literature 1 to 5. Accordingly, an object of the present invention is to provide a thermosensitive recording material and a laminate which solve any drawbacks of the prior arts, which are excellent in color development sensitivity, and which are favorable in heat resistance, moisture resistance, plasticizer resistance, oil resistance, and grease resistance.

### Solution to Problem

The present inventors have made intensive studies, and as a result, have found that a thermosensitive recording material and a laminate in which a thermosensitive recording layer includes a specific diphenylsulfone compound and an analog compound thereof at a specific ratio are each suitable as a color-developer of a thermosensitive recording material which is favorable in sensitivity and favorable in storage stability of a print area, for example, heat resistance, moisture resistance, plasticizer resistance, oil resistance, and grease resistance. Features of the present invention based on the finding are as follows.

[1] A thermosensitive recording material in which a thermosensitive recording layer is provided on a support, wherein the thermosensitive recording layer includes a compound represented by the following formula (1) and a compound represented by the following formula (2), and a content of the compound represented by formula (2) based on a total amount of these compounds is more than 2.0% by weight and 40% by weight or less.

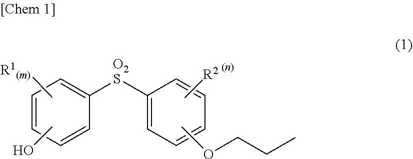

In formula (1), R¹ and R² are optionally the same as or different from each other, and each represents an alkyl group having 1 to 3 carbon atoms, an alkenyl group having 1 to 3 carbon atoms, or a benzyl group, and m and n each independently represent an integer of 0 to 4.

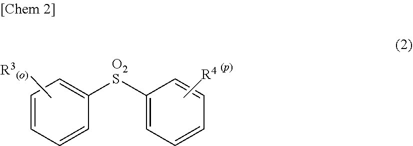

In formula (2), R³ and R⁴ are optionally the same as or different from each other, and each represents an alkyloxy group having 1 to 3 carbon atoms, an alkenyloxy group having 1 to 3 carbon atoms, or a benzyloxy group, and o and p each independently represent an integer of 1 to 3.

[2] The thermosensitive recording material according to [1], wherein the thermosensitive recording layer includes 4-propoxy-4′-hydroxydiphenylsulfone as the compound represented by formula (1).

[3] The thermosensitive recording material according to [1] or [2], wherein the thermosensitive recording layer includes at least one selected from the group consisting of 4,4′-dipropoxydiphenylsulfone, 4,4′-diallyloxydiphenylsulfone, 4,4′-diisopropoxydiphenylsulfone and 4,4′-dibenzyloxydiphenylsulfone, as the compound represented by formula (2).

[4] The thermosensitive recording material according to any one of [1] to [3], wherein the thermosensitive recording layer includes a leuco dye.

[5] The thermosensitive recording material according to [4], including 10 to 200 parts by weight of the leuco dye based on a total of 100 parts by weight of the compound represented by formula (1) and the compound represented by formula (2).

[6] The thermosensitive recording material according to any one of [1] to [5], wherein the thermosensitive recording layer includes a sensitizer.

[7] The thermosensitive recording material according to [6], containing at least one selected from the group consisting of 1,2-di-(3-methylphenoxy)ethane, 1,2-diphenoxyethane, a fatty acid amide having 10 to 21 carbon atoms, β-benzyloxynaphthalene, diphenylsulfone, p-toluenesulfonamide, and oxalic acid-di-p-methylbenzyl ester, as the sensitizer.

[8] A laminate in which a thermosensitive recording layer is provided on a support, wherein the thermosensitive recording layer includes a compound represented by the following formula (1) and a compound represented by the following formula (2), and a content of the compound represented by formula (2) based on a total amount of the compounds is more than 2.0% by weight and 40% by weight or less.

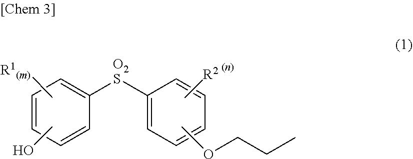

In formula (1), R¹ and R² are optionally the same as or different from each other, and each represents an alkyl group having 1 to 3 carbon atoms, an alkenyl group having 1 to 3 carbon atoms, or a benzyl group, and m and n each independently represent an integer of 0 to 4.

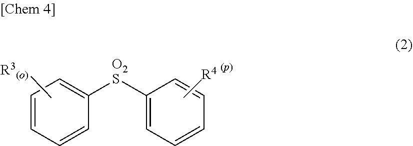

In formula (2), R³ and R⁴ are optionally the same as or different from each other, and each represents an alkyloxy group having 1 to 3 carbon atoms, an alkenyloxy group having 1 to 3 carbon atoms, or a benzyloxy group, and o and p each independently represent an integer of 1 to 3.

[9] The laminate according to [8], wherein the thermosensitive recording layer includes 4-propoxy-4′-hydroxydiphenylsulfone as the compound represented by formula (1).

[10] The laminate according to [8] or [9], wherein the thermosensitive recording layer includes at least one selected from the group consisting of 4,4′-dipropoxydiphenylsulfone, 4,4′-diallyloxydiphenylsulfone, 4,4′-diisopropoxydiphenylsulfone and 4,4′-dibenzyloxydiphenylsulfone, as the compound represented by formula (2).

[11] The laminate according to any one of [8] to [10], wherein the thermosensitive recording layer includes a leuco dye.

[12] The laminate according to [11], including 10 to 200 parts by weight of the leuco dye based on a total of 100 parts by weight of the compound represented by formula (1) and the compound represented by formula (2).

[13] The laminate according to any one of [8] to [12], wherein the thermosensitive recording layer includes a sensitizer.

[14] The laminate according to [13], containing at least one selected from the group consisting of 1,2-di-(3-methylphenoxy) ethane, 1,2-diphenoxyethane, a fatty acid amide having 10 to 21 carbon atoms, β-benzyloxynaphthalene, diphenylsulfone, p-toluenesulfonamide, and oxalic acid-di-p-methylbenzyl ester, as the sensitizer.

### Advantageous Effects of Invention

The present invention provides a thermosensitive recording material and a laminate, both of which are favorable in color development sensitivity and storage stability of a print area, for example, heat resistance, moisture resistance, plasticizer resistance, oil resistance, and grease resistance.

### EMBODIMENTS

Hereinafter, the present invention will be described in detail, but the present invention is not limited to the following description and can be arbitrarily modified and carried out without departing from the gist of the present invention. In the case where the term “to” is used for expression of numerical values or physical property values which are sandwiched before and after the term, in the present invention, such values sandwiched before and after the term are used with being included.

[Thermosensitive Recording Material and Laminate]

The thermosensitive recording material of the present invention is a thermosensitive recording material in which a thermosensitive recording layer is provided on a support, wherein the thermosensitive recording layer includes a compound represented by the following formula (1) (compound (1)) and a compound represented by the following formula (2) (compound (2)), and the content of the compound represented by formula (2) based on the total amount of these compounds is more than 2.0% by weight and 40% by weight or less. The “thermosensitive recording material” in the present invention may be in any form of paper, a film, synthetic paper, a card, and the like as long as the thermosensitive recording layer is provided on the support. The compound (1) usually serves as a color-developer in the thermosensitive recording material of the present invention. The phrase “on the support” here refers to “on at least one surface of the support” and usually refers to “on one surface of the support”. The phrase “provided on the support” means that the relevant layer may be present on at least one portion of the support.

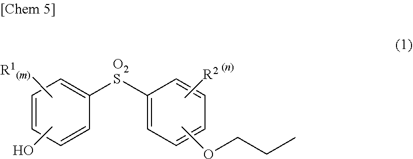

In formula (1), R¹ and R² are optionally the same as or different from each other, and each represents an alkyl group having 1 to 3 carbon atoms, an alkenyl group having 1 to 3 carbon atoms, or a benzyl group, and m and n each independently represent an integer of 0 to 4.

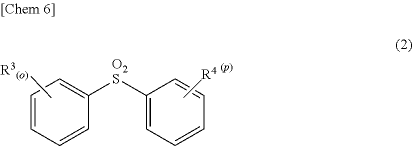

In formula (2), R³ and R⁴ are optionally the same as or different from each other, and each represents an alkyloxy group having 1 to 3 carbon atoms, an alkenyloxy group having 1 to 3 carbon atoms, or a benzyloxy group, and o and p each independently represent an integer of 1 to 3.

The thermosensitive recording material of the present invention, in which the thermosensitive recording layer is provided on the support, may include, if necessary, a topcoat layer (protective layer), an undercoat layer, a backcoat layer, an intermediate coating layer, and the like, as described below. In other words, the thermosensitive recording material of the present invention is usually in the form of a laminate. That is, the laminate of the present invention is a laminate, in which a thermosensitive recording layer is provided on a support, wherein the thermosensitive recording layer includes the compound represented by formula (1) (compound (1)) and the compound represented by formula (2) (compound (2)), and the content of the compound represented by formula (2) based on the total amount of the compounds is more than 2.0% by weight and 40% by weight or less.

The thermosensitive recording material and the laminate of the present invention exert the effects of being excellent in color development sensitivity and favorable in storage characteristics such as heat resistance, moisture resistance, plasticizer resistance, oil resistance, and grease resistance. The reason for this is considered because the compound (2) based on the total amount of the compounds is included in the range of more than 2.0% by weight and 40% by weight or less relative to the compound (1) to thereby allow for an increase in compatibility of the compound (1) and a dye and stabilization of a bonding state between the compound (1) and the dye after color development (namely, in a print area), resulting in favorable storage stability of a print area.

Patent Literature 2 and 3 describe a color-developer of a compound (1) and a thermosensitive recording material using the same, and also mention a compound (2), and the compound (2) has been conventionally recognized as any impurity and, if included in the compound (1), has been removed by any operation such as recrystallization until the amount thereof is equal to or less than the detection limit, before formation and use of such a thermosensitive recording material or a laminate. On the contrary, it has been found for the first time in the present invention that the above excellent effects are obtained by allowing the compound (2), together with the compound (1), to be contained at a specific ratio in the thermosensitive recording layer and thus not to serve as any impurity.

[Thermosensitive Recording Layer]

The thermosensitive recording material and the laminate of the present invention each include a thermosensitive recording layer, and the thermosensitive recording layer includes a compound (1) and a compound (2) at a specific ratio. The thermosensitive recording layer may include, in addition to such compounds, a leuco dye, any color-developer other than the compound (1) and the compound (2), a sensitizer, a stabilizer, a binder, a crosslinking agent, a pigment, a lubricant, other additive, and the like.

<Compound (1) and Compound (2)>

R¹ and R² in formula (1) are optionally the same as or different from each other, and each represents an alkyl group having 1 to 3 carbon atoms, an alkenyl group having 1 to 3 carbon atoms or a benzyl group. Specific examples include a methyl group, an ethyl group, a propyl group, an isopropyl group, a vinyl group, an allyl group, and a benzyl group, preferably a propyl group, an isopropyl group, an allyl group, and a benzyl group, more preferably a propyl group and an allyl group.

In formula (1), m and n are each independently an integer of 0 to 4, and represent the respective numbers of R¹ and R². Herein, “m and n being 0” means that R¹ and R² are not present and four hydrogen atoms are bonded to each aromatic ring. When m is 2 or more, m of R¹(s) are optionally the same as or different from each other. Similarly, when n is 2 or more, n of R²(s) are optionally the same as or different from each other. m is preferably 0 or 1, more preferably 0, namely, a hydrogen atom is bonded. n is preferably an integer of 0 to 2, more preferably 0 or 1, further preferably 0, namely, a hydrogen atom is bonded. When R¹ and R² are present, the positions thereof are not particularly limited.

In the present invention, the thermosensitive recording layer particularly preferably includes 4-propoxy-4′-hydroxydiphenylsulfone as the compound (1).

Next, the compound (2) in use in the present invention is described. R³ and R⁴ in formula (2) are optionally the same as or different from each other, and each represents an alkyloxy group having 1 to 3 carbon atoms, an alkenyloxy group having 1 to 3 carbon atoms, or a benzyloxy group, and specific examples include a methoxy group, an ethoxy group, a propoxy group, an isopropoxy group, an allyloxy group, and a benzyloxy group, preferably a propoxy group, an isopropoxy group, and an allyloxy group, more preferably a propoxy group and an allyloxy group.

In formula (2), o and p are each independently an integer of 0 to 3, and represent the respective numbers of R³ and R⁴. Herein, “o and p being 0” means that R³ and R⁴ are not present and five hydrogen atoms are bonded to each aromatic ring. When o is 2 or more, o of R³(s) are optionally the same as or different from each other. Similarly, when p is 2 or more, p of R⁴(s) are optionally the same as or different from each other. o is preferably 1 or 2, more preferably 1. p is preferably 1 or 2, more preferably 1. When R³ and R⁴ are present, the positions thereof are not particularly limited.

In the present invention, the thermosensitive recording layer preferably includes at least one selected from the group consisting of 4,4′-dipropoxydiphenylsulfone, 4,4′-diallyloxydiphenylsulfone, 4,4′-diisopropoxydiphenylsulfone, and 4,4′-dibenzyloxydiphenylsulfone, as the compound (2). In particular, the thermosensitive recording layer particularly preferably includes at least one selected from the group consisting of 4,4′-dipropoxydiphenylsulfone and 4,4′-diallyloxydiphenylsulfone, as the compound (2).

In the present invention, the content of the compound (2) based on the total amount of the compound (2) and the compound (1) in the thermosensitive recording layer is more than 2.0% by weight, preferably 2.2% by weight or more, more preferably 2.4% by weight or more. On the other hand, the content of the compound (2) in the thermosensitive recording layer is 40% by weight or less, preferably 36% by weight or less, more preferably 32% by weight or less, further preferably 26% by weight or less. The content of the compound (2) is more than the above lower limit to thereby allow storage stability of a print area, for example, heat resistance, oil resistance and grease resistance, to be remarkably favorable. The content of the compound (2) is equal to or less than the upper limit to thereby allow not only deterioration in storage stability of a print area, for example, heat resistance, moisture resistance, plasticizer resistance, oil resistance and grease resistance, but also deterioration in sensitivity to be suppressed.

The present invention also provides a thermosensitive recording material and a laminate each including a support and a thermosensitive recording layer (thermosensitive color-developing layer) provided on the support. In the thermosensitive recording material and the laminate of the present invention, the thermosensitive recording layer contains a colorless or light-colored basic (electron-donating) leuco dye and a color-developer for color development of the basic leuco dye, and the color-developer contains the compound (1). In the thermosensitive recording material and the laminate of the present invention, the compound (1) may be used singly or in combination of two or more kinds thereof.

Hereinafter, any components (other color-developer, basic leuco dye, sensitizer, stabilizer, binder, crosslinking agent, pigment and lubricant, and other additive) other than the compound (1) and the compound (2), which can be used for forming the thermosensitive recording layer, will be sequentially described. All such any components other than the compound (1) and the compound (2) may be used singly or in combination of two or more kinds thereof. Herein, such binder, crosslinking agent, pigment, and the like can be used in not only the thermosensitive recording layer, but also any layer (for example, a topcoat layer (protective layer) described below) other than the thermosensitive recording layer, which can be included in the thermosensitive recording material and the laminate of the present invention.

<Leuco Dye>

In the present invention, a leuco dye is preferably used in the thermosensitive recording layer. Such a leuco dye is usually basic, and can be any of those which are conventionally known in the field of pressure-sensitive or thermosensitive recording paper. The leuco dye is specifically preferably a triphenylmethane-based leuco dye, a fluoran-based leuco dye, a fluorene-based leuco dye, a divinyl leuco dye, or the like. Specific examples of a representative colorless or light-colored dye (dye precursor) are shown below. Such a leuco dye (leuco dye precursor) may be used singly or in combination of two or more kinds thereof. The leuco dye is preferably used in an amount of 10 to 200 parts by eight, more preferably 15 to 150 parts by weight, further preferably 20 to 100 parts by weight based on 100 parts by weight in total of the compound (1) and the compound (2).

Examples of the triphenylmethane-based leuco dye include 3,3-bis(p-dimethylaminophenyl)-6-dimethylaminophthalide [another name: Crystal Violet lactone]; and 3,3-bis(p-dimethylaminophenyl)phthalide [another name: Malachite Green lactone].

Examples of the fluoran-based leuco dye include 3-diethylamino-6-methylfluoran; 3-diethylamino-6-methyl-7-anilinofluoran; 3-diethylamino-6-methyl-7-(o,p-dimethylanilino)fluoran; 3-diethylamino-6-methyl-7-chlorofluoran; 3-diethylamino-6-methyl-7-(m-trifluoromethylanilino)fluoran; 3-diethylamino-6-methyl-7-(o-chloroanilino)fluoran; 3-diethylamino-6-methyl-7-(p-chloroanilino)fluoran; 3-diethylamino-6-methyl-7-(o-fluoroanilino)fluoran; 3-diethylamino-6-methyl-7-(m-methylanilino)fluoran; 3-diethylamino-6-methyl-7-octylanilinofluoran; 3-diethylamino-6-methyl-7-octylaminofluoran; 3-diethylamino-6-methyl-7-benzylaminofluoran; 3-diethylamino-6-methyl-7-dibenzylaminofluoran; 3-diethylamino-6-chloro-7-methylfluoran; 3-diethylamino-6-chloro-7-anilinofluoran; 3-diethylamino-6-chloro-7-p-methylanilinofluoran; 3-diethylamino-6-ethoxyethyl-7-anilinofluoran; 3-diethylamino-7-methylfluoran; 3-diethylamino-7-chlorofluoran; 3-diethylamino-7-(m-trifluoromethylanilino)fluoran; 3-diethylamino-7-(o-chloroanilino)fluoran; 3-diethylamino-7-(p-chloroanilino)fluoran; 3-diethylamino-7-(o-fluoroanilino)fluoran; 3-diethylamino-benzo[a]fluoran; 3-diethylamino-benzo[c]fluoran; 3-dibutylamino-6-methyl-fluoran; 3-dibutylamino-6-methyl-7-anilinofluoran; 3-dibutylamino-6-methyl-7-(o,p-dimethylanilino)fluoran; 3-dibutylamino-6-methyl-7-(o-chloroanilino)fluoran; 3-dibutylamino-6-methyl-7-(p-chloroanilino)fluoran; 3-dibutylamino-6-methyl-7-(o-fluoroanilino)fluoran; 3-dibutylamino-6-methyl-7-(m-trifluoromethylanilino)fluoran; 3-dibutylamino-6-methyl-chlorofluoran; 3-dibutylamino-6-ethoxyethyl-7-anilinofluoran; 3-dibutylamino-6-chloro-7-anilinofluoran; 3-dibutylamino-6-methyl-7-p-methylanilinofluoran; 3-dibutylamino-7-(o-chloroanilino)fluoran; 3-dibutylamino-7-(o-fluoroanilino)fluoran; 3-di-pentylamino-6-methyl-7-anilinofluoran; 3-di-pentylamino-6-methyl-7-(p-chloroanilino)fluoran; 3-di-pentylamino-7-(m-trifluoromethylanilino)fluoran; 3-di-pentylamino-6-chloro-7-anilinofluoran; 3-di-pentylamino-7-(p-chloroanilino)fluoran; 3-pyrrolizino-6-methyl-7-anilinofluoran; 3-piperidino-6-methyl-7-anilinofluoran; 3-(N-methyl-N-propylamino)-6-methyl-7-anilinofluoran; 3-(N-methyl-N-cyclohexylamino)-6-methyl-7-anilinofluoran; 3-(N-ethyl-N-cyclohexylamino)-6-methyl-7-anilinofluoran; 3-(N-ethyl-N-cyclohexylamino)-6-methyl-7-(p-chloroanilino)fluoran; 3-(N-ethyl-p-toluidino)-6-methyl-7-anilinofluoran; 3-(N-ethyl-N-isoamylamino)-6-methyl-7-anilinofluoran; 3-(N-ethyl-N-isoamylamino)-6-chloro-7-anilinofluoran; 3-(N-ethyl-N-tetrahydrofurfurylamino)-6-methyl-7-anilinofluoran; 3-(N-ethyl-N-isobutylamino)-6-methyl-7-anilinofluoran; 3-(N-ethyl-N-ethoxypropylamino)-6-methyl-7-anilinofluoran; 3-cyclohexylamino-6-chlorofluoran; 2-(4-oxahexyl)-3-dimethylamino-6-methyl-7-anilinofluoran; 2-(4-oxahexyl)-3-diethylamino-6-methyl-7-anilinofluoran; 2-(4-oxahexyl)-3-dipropylamino-6-methyl-7-anilinofluoran; 2-methyl-6-p-(p-dimethylaminophenyl)aminoanilinofluoran; 2-methoxy-6-p-(p-dimethylaminophenyl)aminoanilinofluoran; 2-chloro-3-methyl-6-p-(p-phenylaminophenyl)aminoanilinofluoran; 2-chloro-6-p-(p-dimethylaminophenyl)aminoanilinofluoran; 2-nitro-6-p-(p-diethylaminophenyl)aminoanilinofluoran; 2-amino-6-p-(p-diethylaminophenyl)aminoanilinofluoran; 2-diethylamino-6-p-(p-diethylaminophenyl)aminoanilinofluoran; 2-phenyl-6-methyl-6-p-(p-phenylaminophenyl)aminoanilinofluoran; 2-benzyl-6-p-(p-phenylaminophenyl)aminoanilinofluoran; 2-hydroxy-6-p-(p-phenylaminophenyl)aminoanilinofluoran; 3-methyl-6-p-(p-dimethylaminophenyl)aminoanilinofluoran; 3-diethylamino-6-p-(p-diethylaminophenyl)aminoanilinofluoran; 3-diethylamino-6-p-(p-dibutylaminophenyl)aminoanilinofluoran; and 2,4-dimethyl-6-[(4-dimethylamino)anilino]fluoran.

Examples of the fluorene-based leuco dye include 3,6,6′-tris(dimethylamino)spiro[fluorene-9,3′-phthalide]; and 3,6,6′-tris(diethylamino)spiro[fluorene-9,3′-phthalide].

Examples of the divinyl leuco dye include 3,3-bis[2-(p-dimethylaminophenyl)-2-(p-methoxyphenyl)ethenyl]-4,5,6,7-tetrabromophthalide; 3,3-bis[2-(p-dimethylaminophenyl)-2-(p-methoxyphenyl)ethenyl]-4,5,6,7-tetrachlorophthalide; 3,3-bis[1,1-bis(4-pyrrolizinophenyl)ethylen-2-yl]-4,5,6,7-tetrabromophthalide; and 3,3-bis[1-(4-methoxyphenyl)-1-(4-pyrrolizinophenyl)ethylen-2-yl]-4,5,6,7-tetrachlorophthalide.

Examples of other leuco dye include 3-(4-diethylamino-2-ethoxyphenyl)-3-(1-ethyl-2-methylindol-3-yl)-4-azaphthalide; 3-(4-diethylamino-2-ethoxyphenyl)-3-(1-octyl-2-methylindol-3-yl)-4-azaphthalide; 3-(4-cyclohexylethylamino-2-methoxyphenyl)-3-(1-ethyl-2-methylindol-3-yl)-4-azaphthalide; 3,3-bis(1-ethyl-2-methylindol-3-yl)phthalide; 3,6-bis(diethylamino)fluoran-γ-(3′-nitro)anilinolactam; 3,6-bis(diethylamino)fluoran-γ-(4′-nitro)anilinolactam; 1,1-bis[2′,2′,2″,2″-tetrakis-(p-dimethylaminophenyl)-ethenyl]-2,2-dinitrileethane; 1,1-bis[2′,2′,2″,2″-tetrakis-(p-dimethylaminophenyl)-ethenyl]-2-β-naphthoylethane; 1,1-bis[2′,2′,2″,2″-tetrakis(p-dimethylaminophenyl)ethenyl]-2,2-diacetylethane; and bis[2,2,2′,2′-tetrakis(p-dimethylaminophenyl)ethenyl]-methylmalonic acid dimethyl ester.

<Other Color-Developer>

The thermosensitive recording layer may contain any color-developer (hereinafter, designated as “other color-developer”) other than the compound (1) in the present invention as long as the effects of the present invention are not impaired. Such other color-developer here used may be any of those conventionally known in the field of pressure-sensitive or thermosensitive recording paper, is not particularly limited, and is preferably an electron-accepting color-developer. Such other color-developer may be used singly or in combination of two or more kinds thereof. In the case where such other color-developer is used, the amount thereof used is preferably 1 to 100 parts by weight, more preferably 1 to 70 parts by weight, further preferably 1 to 50 parts by weight based on a total of 100 parts by weight of the compound (1) and the compound (2).

Such other color-developer can be used to thereby provide a thermosensitive recording material and a laminate which can not only keep high color development sensitivity, but also are further enhanced in image storage stability with heat resistance, moisture resistance, and water-resistance.

Such other color-developer which can be used in the present invention is any of those conventionally known in the field of pressure-sensitive or thermosensitive recording paper, and is not particularly limited. Such other color-developer is preferably a bisphenolic compound, a urea-based compound, and a novolac type phenolic compound.

Examples of the bisphenolic compound include 4,4′-isopropylidenediphenol, 2,2′-bis(4-hydroxy-3-methylphenyl)propane, 1,1-bis(4-hydroxyphenyl)cyclohexane, 2,2-bis(4-hydroxyphenyl)-4-methylpentane, 4,4′-dihydroxydiphenyl sulfide, di(4-hydroxy-3-methylphenyl)sulfide, 2,2′-thiobis(3-tert-octylphenol), 2,2′-thiobis(4-tert-octylphenol), 4,4′-dihydroxydiphenylsulfone, 2,4′-dihydroxydiphenylsulfone, 4-hydroxy-4′-propoxydiphenylsulfone, 4-hydroxy-4′-isopropoxydiphenylsulfone, 4-hydroxy-4′-allyloxydiphenylsulfone, bis(3-allyl-4-hydroxyphenyl)sulfone, 4-hydroxyphenyl-4′-benzyloxyphenylsulfone, 3,4-dihydroxyphenyl-4′-methylphenylsulfone, a bisphenolsulfone crosslinking type compound described in JP 3913820 B, and a bisphenolsulfone derivative described in JP 4004289 B.

Examples of the urea-based compound include 4,4′-bis(3-(phenoxycarbonylamino)methylphenylureido)diphenylsulfone, and N-(p-toluenesulfonyl)-N′-(3-p-toluenesulfonyloxyphenyl)urea and a derivative thereof described in JP 4601174 B.

Examples of the novolac type phenolic compound include a phenol-formalin condensate described in WO 02/098674.

Examples include, in addition to the compounds exemplified above, inorganic acidic substances such as activated white earth, attapulgite, colloidal silica and aluminum silicate, hydroquinone monobenzyl ether, benzyl 4-hydroxybenzoate, an aminobenzenesulfonamide derivative described in JP H8-59603 A, bis(4-hydroxyphenylthioethoxy)methane, 1,5-di(4-hydroxyphenylthio)-3-oxapentane, butyl bis(p-hydroxyphenyl)acetate, methyl bis(p-hydroxyphenyl)acetate, 1,1-bis(4-hydroxyphenyl)-1-phenylethane, 1,4-bis[α-methyl-α-(4′-hydroxyphenyl)ethyl]benzene, 1,3-bis[α-methyl-α-(4′-hydroxyphenyl)ethyl]benzene, any compound described in WO 02/081229 and JP 2002-301873 A, thiourea compounds such as N,N′-di-m-chlorophenylthiourea, p-chlorobenzoic acid, stearyl gallate, zinc bis[4-(octyloxycarbonylamino)salicylate]-dihydrate, aromatic carboxylic acids such as 4-[2-(p-methoxyphenoxy)ethyloxy]salicylic acid, 4-[3-(p-tolylsulfonyl)propyloxy]salicylic acid and 5-[p-(2-p-methoxyphenoxyethoxy)cumyl]salicylic acid, and salts of such aromatic carboxylic acids with polyvalent metal such as zinc, magnesium, aluminum, calcium, titanium, manganese, tin and nickel, as well as an antipyrine complex of zinc thiocyanate, and a complex zinc salt of terephthalaldehydic acid with other aromatic carboxylic acid. Examples include metal chelate complexes such as a metal double salt of a higher fatty acid and a polyhydroxy aromatic compound described in JP H10-258577 A.

Among other color-developers described above, preferable are 4,4′-dihydroxydiphenylsulfone, 2,4′-dihydroxydiphenylsulfone, 4-hydroxy-4′-propoxydiphenylsulfone, 4-hydroxy-4′-isopropoxydiphenylsulfone, 4-hydroxy-4′-allyloxydiphenylsulfone, bis(3-allyl-4-hydroxyphenyl)sulfone, 4,4′-isopropylidenediphenol, 2,2′-bis(4-hydroxy-3-methylphenyl)propane, a diphenylsulfone crosslinking type compound described in JP 3913820 B, a diphenylsulfone derivative described in JP 4004289 B, a phenol-formalin condensate described in WO 02/098674, 4,4′-bis(3-(phenoxycarbonylamino)methylphenylureido)diphenylsulfone, and N-(p-toluenesulfonyl)-N′-(3-p-toluenesulfonyloxyphenyl)urea and a derivative thereof described in JP 4601174 B, and more preferable are 4,4′-dihydroxydiphenylsulfone, 2,4′-dihydroxydiphenylsulfone, 4-hydroxy-4′-propoxydiphenylsulfone, 4-hydroxy-4′-isopropoxydiphenylsulfone, 4-hydroxy-4′-allyloxydiphenylsulfone, bis(3-allyl-4-hydroxyphenyl)sulfone, a bisphenolsulfone crosslinking type compound described in JP 3913820 B, and a bisphenolsulfone derivative described in JP 4004289 B. Such other color-developer can be used to thereby allow the thermosensitive recording material and the laminate to not only keep color development sensitivity, but also be enhanced in image storage stability (heat resistance, plasticizer resistance, moisture resistance, and water resistance) and the like.

<Sensitizer>

A known sensitizer may also be used in the present invention. The sensitizer here used is not particularly limited, and can be, for example, 1,2-di-(3-methylphenoxy) ethane, β-benzyloxynaphthalene, a fatty acid amide having 10 to 21 carbon atoms (for example, stearic acid amide and palmitic acid amide), ethylene bisamide, montanic acid wax, polyethylene wax, p-benzylbiphenyl, diphenylsulfone, 4-biphenyl-p-tolyl ether, m-terphenyl, 1,2-diphenoxyethane, dibenzyl oxalate, di(p-chlorobenzyl) oxalate, di(p-methylbenzyl) oxalate, dibenzyl terephthalate, benzyl p-benzyloxybenzoate, di-p-tolyl carbonate, phenyl-α-naphthyl carbonate, 1,4-diethoxynaphthalene, 1-hydroxy-2-naphthoic acid phenyl ester, o-xylene-bis-(phenyl ether), 4-(m-methylphenoxymethyl)biphenyl, 4,4′-ethylenedioxy-bis-benzoic acid dibenzyl ester, dibenzoyloxymethane, 1,2-di(3-methylphenoxy)ethylene, bis[2-(4-methoxy-phenoxy)ethyl]ether, methyl p-nitrobenzoate, or phenyl p-toluenesulfonate. In particular, 1,2-di-(3-methylphenoxy)ethane, 1,2-diphenoxyethane, a fatty acid amide having 10 to 21 carbon atoms (for example, stearic acid amide and palmitic acid amide), β-benzyloxynaphthalene, diphenylsulfone, p-toluenesulfonamide and oxalic acid-di-p-methylbenzyl ester are preferable, and 1,2-di-(3-methylphenoxy)ethane exhibiting high color development sensitivity even at a low energy is particularly preferable. Such a sensitizer may be used singly or in combination of two or more kinds thereof. In the case where the sensitizer is used, the amount thereof used is preferably 25 to 250 parts by weight, more preferably 50 to 150 parts by weight based on a total of 100 parts by weight of the compound (1) and the compound (2).

<Stabilizer>

In the present invention, a stabilizer may also be used in the thermosensitive recording layer in order to enhance image storage stability of the thermosensitive recording material and the laminate. Such a stabilizer refers to one having the effect of enhancing image storage stability. Examples of the stabilizer include a hindered phenolic compound, an ultraviolet absorber (for example, a benzophenone-based compound and a triazole-based compound), and an antioxidant. In particular, a hindered phenolic compound is preferable in that image storage stability of a recording area (heat resistance, moisture resistance, water resistance, plasticizer resistance, and the like) is enhanced.

The hindered phenolic compound is a compound having 1 or more and 15 or less, preferably 2 or more and 6 or less hydroxyphenyl groups in one molecule. The molecular weight of the hindered phenolic compound is usually 200 or more and 2000 or less, preferably 250 or more and 1800 or less, more preferably 300 or more and 1500 or less. The melting point of the hindered phenolic compound is preferably 100° C. or more and 300° C. or less.

The hindered phenolic compound is preferably one that in at least one of the hydroxyphenyl groups contained in the hindered phenolic compound, any of the carbon atoms at the 2-position or 6-position is bonded to a hydrogen atom (namely, no substituent is present at the 2-position or 6-position) under the assumption that the position of a phenolic hydroxyl group defined as the 1-position.

Specific examples of the hindered phenolic compound include tris(hydroxyphenyl)alkane and a 1,1,3-tris-substituted butane compound described in JP S39-4469 B or JP S56-40629 A. Two or more of such compounds may also be used in combination.

The hindered phenolic compound may be used singly or in combination of two or more kinds thereof. In the case where the hindered phenolic compound is used in the thermosensitive recording material and the laminate of the present invention, the content thereof is preferably 1 to 100 parts by weight, more preferably 1 to 70 parts by weight, further preferably 1 to 50 parts by weight based on a total of 100 parts by weight of the compound (1) and the compound (2). If the content of the hindered phenolic compound is less than the range, a recording area can be deteriorated in moisture resistance, water resistance, and heat resistance, and color development in a blank area due to heating cannot be suppressed. If the content is more than the range, color development sensitivity can be deteriorated and plasticizer resistance of a recording area can be deteriorated.

<Binder>

A binder is preferably used for forming the thermosensitive recording layer. Examples of the binder include completely saponified polyvinyl alcohol, partially saponified polyvinyl alcohol, acetoacetylated polyvinyl alcohol, carboxy-modified polyvinyl alcohol, amide-modified polyvinyl alcohol, sulfonic acid-modified polyvinyl alcohol, butyral-modified polyvinyl alcohol, olefin-modified polyvinyl alcohol, nitrile-modified polyvinyl alcohol, pyrrolidone-modified polyvinyl alcohol, silicone-modified polyvinyl alcohol, other modified polyvinyl alcohol, hydroxyethylcellulose, methylcellulose, ethylcellulose, carboxymethylcellulose, polystyrene, styrene copolymers such as a styrene-maleic anhydride copolymer and a styrene-butadiene copolymer, cellulose derivatives such as ethylcellulose and acetylcellulose, casein, gum arabic, oxidized starch, etherified starch, dialdehyde starch, esterified starch, polyvinyl chloride, polyvinyl acetate, polyacrylamide, polyacrylate, polyvinyl butyral, a polyamide resin, a silicone resin, a petroleum resin, a terpene resin, a ketone resin, and a coumarone resin. The amount of the binder used, is appropriately about 5 to 25% by weight of the solid content of the thermosensitive recording layer.

The binder is generally used in the form of a solution, an emulsion, a dispersion liquid, a paste, or a combination thereof. Examples of the solvent for the solution, the emulsion or the dispersion liquid, or the medium for the paste include water, alcohols, ketones, esters and hydrocarbons.

<Crosslinking Agent>

Examples of the crosslinking agent include glyoxal, methylolmelamine, a melamine formaldehyde resin, a melamine urea resin, a polyamine epichlorohydrin resin, a polyamide epichlorohydrin resin, potassium persulfate, ammonium persulfate, sodium persulfate, ferric chloride, magnesium chloride, borax, boric acid, alum, and ammonium chloride. In the case where the crosslinking agent is used, the amount thereof used is preferably 0.5 to 500 parts by weight based on a total of 100 parts by weight of the compound (1) and the compound (2).

<Pigment>

Examples of the pigment include inorganic or organic pigments such as silica (except for colloidal silica), calcium carbonate, kaolin, calcined kaolin, diatomous earth, talc, titanium oxide, and aluminum hydroxide. In the case where the pigment is used, the amount thereof used is preferably 25 to 1000 parts by weight based on a total of 100 parts by weight of the compound (1) and the compound (2).

<Lubricant>

Examples of the lubricant include fatty acid metal salts such as zinc stearate and calcium stearate, waxes, and silicone resins. In the case where the lubricant is used, the amount thereof used is preferably 0.5 to 500 parts by weight based on a total of 100 parts by weight of the compound (1) and the compound (2).

<Other Additives>

Examples of such other additives include a dispersant, a defoamer and a fluorescent dye. In the case where such other additives being used, the amount thereof used is preferably 0.5 to 500 parts by weight based on a total of 100 parts by weight of the compound (1) and the compound (2).

[Support]

The shape, the structure, the size, the material, and the like of the support in use in the thermosensitive recording material and the laminate of the present invention are not particularly limited, and can be appropriately selected depending on the object. Examples of the shape of the support include a sheet shape, a roll shape, and a plate shape. The support may have a monolayer structure or a layered structure. The size of the support can be appropriately selected depending on the applications of objective thermosensitive recording material and laminate, and the like. Examples of the material of the support include a plastic film, synthetic paper, high-quality paper, waste paper pulp, recycled paper, machine-glazed paper, greaseproof paper, coat paper, art paper, cast-coat paper, light-weight coated paper, resin laminated paper, and release paper. A composite sheet obtained by a combination of such materials may also be used as the support.

The thickness of the support is not particularly limited, can be appropriately selected depending on the object, and is preferably 30 to 2,000 μm, more preferably 50 to 1,000 μm.

[Topcoat Layer (Protective Layer)]

A topcoat layer (protective layer) may be provided on the thermosensitive recording layer in the thermosensitive recording material and the laminate of the present invention. In general, in the case where a topcoat layer (protective layer) is provided on a thermosensitive recording layer to thereby enhance image storage stability in a thermosensitive recording material and a laminate, color development sensitivity at low energy is deteriorated. However, the thermosensitive recording material and the laminate of the present invention, in which the compound (1) is used as a color-developer, are thus favorable in color development sensitivity at low energy even in the case where a topcoat layer (protective layer) is provided on the thermosensitive recording layer. The types and amounts of various components in use in the topcoat layer (protective layer) are determined according to any required performance and/or recording suitability, and are not particularly limited.

[Undercoat Layer/Backcoat Layer/Intermediate Coating Layer]

An undercoat layer mainly including a pigment and a binder can also be provided between the support and the thermosensitive recording layer in the thermosensitive recording material and the laminate of the present invention, for the purpose of further enhancing color development sensitivity. A backcoat layer may also be provided on an opposite surface of the support to the one that exists thermosensitive recording layer, in order to achieve correction of curling of the thermosensitive recording material and the laminate of the present invention. Examples of one aspect with respect to each layer in the thermosensitive recording material and the laminate of the present invention include, but not limited thereto, an aspect where layering is made in the order of topcoat (protective layer)/thermosensitive recording layer/undercoat layer/support/backcoat layer.

Furthermore, any intermediate coating layer may also be formed between the support and the undercoat layer, between the undercoat layer and the thermosensitive recording layer, between the thermosensitive recording layer and the topcoat layer (protective layer), and/or between the support and the backcoat layer.

[Method of Producing Thermosensitive Recording Material and Laminate]

The thermosensitive recording material and the laminate of the present invention can be each conventionally produced by coating at least a portion on at least one surface of the support with a coating liquid containing the leuco dye, the compound (1) and the compound (2), and, if necessary, other color-developer, the sensitizer, the stabilizer, and the like, and drying the resultant to thereby form the thermosensitive recording layer. Such coating with the coating liquid can be made according to a well-known conventional art. A coating unit is not particularly limited, and, for example, an off-machine coater or an on-machine coater can be used which is equipped with any of various coaters such as an air knife coater, a rod blade coater, a bent blade coater, a bevel blade coater, a roll coater, and a curtain coater.

The coating liquid for formation of the thermosensitive recording layer can be formed by, for example, blending the compound (1) and the compound (2), and, if necessary, the leuco dye, other color-developer, the hindered phenolic compound, the sensitizer, and the like, atomizing the resultant until a particle size of several microns or less is achieved, by a pulverizer such as a ball mill, an attriter or a sand grinder, or an appropriate emulsification apparatus, and thereafter adding the binder or the like thereto. The solvent in use in the coating liquid can be water, an alcohol, or the like. The solid content of the coating liquid is usually 20 to 40% by weight.

The amount of coating with the thermosensitive recording layer can be appropriately selected depending on the composition of the layer, the applications of the thermosensitive recording material and the laminate, and the like, and is usually in the range from 1 to 20 g/m², preferably 2 to 12 g/m² on a dry weight.

The topcoat layer (protective layer), the undercoat layer, the backcoat layer, and the intermediate coating layer can also be each formed by coating with a coating liquid including each constituent component and drying in the same manner as in the thermosensitive recording layer. The thermosensitive recording material and the laminate of the present invention, in which the respective layers are formed, can be subjected to any treatment (for example, a smoothing treatment with a supercalender or the like) known in the art.

[Applications of Thermosensitive Recording Material and Laminate]

The thermosensitive recording material and the laminate of the present invention can be suitably used in any applications such as paper, a film, an IC card and a friction ballpoint pen.

### EXAMPLES

Hereinafter, the present invention will be specifically described with reference to Examples, but the scope of the present invention is not intended to be limited to such Examples.

A thermosensitive recording layer (thermosensitive color-developing layer) was formed by using paper where an underlayer was provided on one surface of a support, in Examples and Comparative Examples below. Unless particularly noted, “part(s)” and “%” in the following Examples and Comparative Examples mean “part(s) by weight” and “% by weight”, respectively.

[Preparation of Coating Liquid for Thermosensitive Recording Layer]

Respective liquid A to liquid G described below were prepared. The liquid A to liquid D were each wet pulverized by Bead mill/LMZ manufactured by Ashizawa Finetech Ltd., until the average particle size of each component reached 0.5 μm. The average particle size here corresponded to an average size on a volume basis distribution, and was measured by a laser diffraction/scattering particle size distribution measuring apparatus (Microtrac MT3000II) manufactured by Nikkiso Co., Ltd.

<Liquid A>
4-Propoxy-4′-hydroxydiphenylsulfone (trade name “TOMILAC KN” manufactured by Mitsubishi Chemical Corporation): 40.0 parts
10% aqueous solution of polyvinyl alcohol (“GL-03” manufactured by NIPPON GOHSEI): 50.0 parts
Water: 10.0 parts

<Liquid B>
4,4′-Dipropoxydiphenylsulfone: 40.0 parts
10% aqueous solution of polyvinyl alcohol (“GL-03” manufactured by NIPPON GOHSEI): 50.0 parts
Water: 10.0 parts

<Liquid C>
3-Dibutylamino-6-methyl-7-anilinofluoran (trade name “ODB-2” manufactured by Yamamoto Chemicals Inc.): 36.5 parts
10% aqueous solution of polyvinyl alcohol (“L-3266” manufactured by NIPPON GOHSEI): 60.0 parts
Water: 3.5 parts

<Liquid D>
1,2-Di-(3-methylphenoxy)ethane (trade name “KS-232” manufactured by SANKOSHA CO., LTD.): 40.0 parts
10% aqueous solution of polyvinyl alcohol (“L-3266” manufactured by NIPPON GOHSEI): 50.0 parts
Water: 10.0 parts

<Liquid E>
Calcium carbonate dispersion liquid having a solid content of 60% (trade name “TAMAPEARL TP-123CS”) manufactured by OKUTAMA KOGYO CO., LTD.

<Liquid F>
36% Zinc stearate dispersion liquid (trade name “Hydrin Z-8-36”) manufactured by CHUKYO YUSHI CO., LTD.

<Liquid G>
10% aqueous polyvinyl alcohol solution (10% aqueous solution of “GOHSENOL NH-18”) manufactured by NIPPON GOHSEI)

### Examples 1-1 to 1-8 and Comparative Examples 1-1 to 1-3

Examples 1-1 to 1-8 and Comparative Examples 1-1 to 1-3 below were performed in order to confirm the difference in effect due to the amounts of the compound (1) and the compound (2) blended.

### Example 1-1

Respective liquids were mixed at the following proportions to thereby prepare a coating liquid for a thermosensitive recording layer.

Liquid A: 18.42 parts

Liquid B: 0.47 parts

Liquid C: 10.00 parts

Liquid D: 18.89 parts

Liquid E: 29.75 parts

Liquid F: 9.95 parts

Liquid G: 32.64 parts

Next, paper where an underlayer as a support was provided on one surface of high-quality paper was coated with the coating liquid for a thermosensitive recording layer, having the above composition, so that the dry weight of a thermosensitive recording layer was 6 g/m², and the resultant was dried by a fan dryer to thereby form a thermosensitive recording layer. The layer was smoothed under application of a pressure of 1 kgf/cm² by a supercalender, thereby providing a thermosensitive recording material (laminate).

### Example 1-2

A thermosensitive recording material (laminate) was produced in the same manner as in Example 1-1 except that it was changed to 17.95 parts of the liquid A and 0.94 parts of the liquid B.

### Example 1-3

A thermosensitive recording material (laminate) was produced in the same manner as in Example 1-1 except that it was changed to 17.47 parts of the liquid A and 1.42 parts of the liquid B.

### Example 1-4

A thermosensitive recording material (laminate) was produced in the same manner as in Example 1-1 except that it was changed to 17.00 parts of the liquid A and 1.89 parts of the liquid B.

### Example 1-5

A thermosensitive recording material (laminate) was produced in the same manner as in Example 1-1 except that it was changed to 16.06 parts of the liquid A and 2.83 parts of the liquid B.

### Example 1-6

A thermosensitive recording material (laminate) was produced in the same manner as in Example 1-1 except that it was changed to 15.11 parts of the liquid A and 3.78 parts of the liquid B.

### Example 1-7

A thermosensitive recording material (laminate) was produced in the same manner as in Example 1-1 except that it was changed to 14.17 parts of the liquid A and 4.72 parts of the liquid B.

### Example 1-8

A thermosensitive recording material (laminate) was produced in the same manner as in Example 1-1 except that it was changed to 13.22 parts of the liquid A and 5.67 parts of the liquid B.

### Comparative Example 1-1

A thermosensitive recording material (laminate) was produced in the same manner as in Example 1-1 except that no liquid B was used and it was changed to 18.89 parts of the liquid A.

### Comparative Example 1-2

A thermosensitive recording material (laminate) was produced in the same manner as in Example 1-1 except that it was changed to 9.445 parts of the liquid A and 9.445 parts of the liquid B.

### Comparative Example 1-3

A thermosensitive recording material (laminate) was produced in the same manner as in Example 1-1 except that no liquid B was used and 18.89 parts of a liquid where 4-propoxy-4′-hydroxydiphenylsulfone as the liquid A was changed to 4-allyloxy-4′-hydroxydiphenylsulfone was used.

[Evaluation of Thermosensitive Recording Material (Laminate)]

Each of the thermosensitive recording materials (laminates) obtained in Examples and Comparative Examples was evaluated as follows.

<Color Development Sensitivity>

A gradation pattern was printed by using a thermosensitive printer (TH-M2/PS) manufactured by Ohkura Electric Co., Ltd., and the image density and the density of a blank sheet area at an applied energy of 0.36 mJ/dot were measured with an eXact densitometer manufactured by X-Rite, Incorporated. The results are shown in Table-1. A higher value with respect to the results in the present test indicates more favorable color development sensitivity.

<Heat Resistance>

After a thermosensitive recording material where a checkerboard pattern was printed at an applied energy of 0.36 mJ/dot by use of a thermosensitive printer (TH-M2/PS) manufactured by Ohkura Electric Co., Ltd. was left to still stand at 90° C. and 100° C. for 1 hour, the print density of a print area was measured with an eXact densitometer manufactured by X-Rite, Incorporated. The results are shown in Table-1. A higher value with respect to the results in the present test indicates that heat resistance is more favorable.

<Oil Resistance>

After one droplet of vegetable oil (manufactured by Nisshin OilliO Group, Ltd.) was placed on a print area of a thermosensitive recording material where a checkerboard pattern was printed at an applied energy of 0.36 mJ/dot by use of a thermosensitive printer (TH-M2/PS) manufactured by Ohkura Electric Co., Ltd., and lightly wiped up and left to still stand at 20° C. for 24 hours, the print density of a print area was measured with an eXact densitometer manufactured by X-Rite, Incorporated. The results are shown in Table-1. A higher value with respect to the results in the present test indicates more favorable oil resistance.

<Grease Resistance>

After one droplet of Skin Milk (manufactured by Nivea-Kao Co., Ltd.) was placed on a print area of a thermosensitive recording material where a checkerboard pattern was printed at an applied energy of 0.36 mJ/dot by use of a thermosensitive printer (TH-M2/PS) manufactured by Ohkura Electric Co., Ltd., and lightly wiped up and left to still stand at 20° C. for 24 hours, the print density of a print area was measured with an eXact densitometer manufactured by X-Rite, Incorporated. The results are shown in Table-1. A higher value with respect to the results in the present test indicates more favorable grease resistance.

TABLE 1

| | | | Color | | | | |
|---|---|---|---|---|---|---|---|
| | | | development | | | Oil | Grease |
| | | | sensitivity | Heat resistance | | resistance | resistance |
| | Content of | Content of | 0.36 mJ/dot | 90° C.-1 h | 100° C.-1 h | r.t.-24 h | r.t.-24 h |
| | compound (1) | compound (2) | Image | Image | Image | Image | Image |
| | [% by weight] | [% by weight] | density | density | density | density | density |
| Comparative | 100 | — | 1.37 | 0.71 | 0.83 | 0.28 | 0.25 |
| Example 1-1 | | | | | | | |
| Example 1-1 | 97.5 | 2.5 | 1.35 | 1.14 | 1.14 | 0.44 | 0.35 |
| Example 1-2 | 95 | 5 | 1.35 | 1.21 | 1.21 | 0.65 | 0.56 |
| Example 1-3 | 92.5 | 7.5 | 1.35 | 1.22 | 1.22 | 0.78 | 0.85 |
| Example 1-4 | 90 | 10 | 1.34 | 1.23 | 1.22 | 0.80 | 1.16 |
| Example 1-5 | 85 | 15 | 1.32 | 1.20 | 1.19 | 0.77 | 1.25 |
| Example 1-6 | 80 | 20 | 1.30 | 1.18 | 1.17 | 0.74 | 1.24 |
| Example 1-7 | 75 | 25 | 1.30 | 1.18 | 1.17 | 0.64 | 1.08 |
| Example 1-8 | 70 | 30 | 1.27 | 1.15 | 1.13 | 0.48 | 0.87 |
| Comparative | 50 | 50 | 1.13 | 0.97 | 0.95 | 0.22 | 0.34 |
| Example 1-2 | | | | | | | |
| Comparative | — | — | 1.38 | 0.45 | 0.59 | 0.28 | 0.26 |
| Example 1-3 | | | | | | | |

As clear from Table-1, Examples 1-1 to 1-8 were favorable in storage characteristics of a print area, such as heat resistance, oil resistance and grease resistance, in comparison of Examples 1-1 to 1-8 each corresponding to the thermosensitive recording material of the present invention with Comparative Example 1-1 or 1-3 including no compound (2) and Comparative Example 1-2 including a larger amount of the compound (2) than the present invention.

### Examples 2-1 to 2-6 and Comparative Examples 2-1 to 2-8

Examples 2-1 to 2-6 and Comparative Examples 2-1 to 2-8 below were performed in order to confirm the difference in effect due to the types of the compound (1) and the compound (2).

### Example 2-1

Respective liquids were mixed at the following proportions to thereby prepare a coating liquid for a thermosensitive recording layer.

Liquid A: 18.42 parts

Liquid B: 0.47 parts

Liquid C: 10.00 parts

Liquid D: 18.89 parts

Liquid E: 29.75 parts

Liquid F: 9.95 parts

Liquid G: 32.64 parts

Next, high-quality paper as a support, where no undercoat layer was provided, was coated with the coating liquid for a thermosensitive recording layer, having the above composition, so that the dry weight of a thermosensitive recording layer was 6 g/m², and the resultant was dried by a fan dryer to thereby form a thermosensitive recording layer. The layer was smoothed under application of a pressure of 1 kgf/cm² by a supercalender, thereby providing a thermosensitive recording material (laminate).

### Example 2-2

A thermosensitive recording material (laminate) was produced in the same manner as in Example 2-1 except that it was changed to 16.06 parts of the liquid A and 2.83 parts of the liquid B.

### Example 2-3

A thermosensitive recording material (laminate) was produced in the same manner as in Example 2-1 except that it was changed to 13.22 parts of the liquid A and 5.67 parts of the liquid B.

### Example 2-4

A thermosensitive recording material (laminate) was produced in the same manner as in Example 2-1 except that 4,4′-dipropoxydiphenylsulfone as the liquid B was changed to 4,4′-diallyloxydiphenylsulfone.

### Example 2-5

A thermosensitive recording material (laminate) was produced in the same manner as in Example 2-2 except that 4,4′-dipropoxydiphenylsulfone as the liquid B was changed to 4,4′-diallyloxydiphenylsulfone.

### Example 2-6

A thermosensitive recording material (laminate) was produced in the same manner as in Example 2-3 except that 4,4′-dipropoxydiphenylsulfone as the liquid B was changed to 4,4′-diallyloxydiphenylsulfone.

### Comparative Example 2-1

A thermosensitive recording material (laminate) was produced in the same manner as in Example 2-1 except that no liquid B was used and it was changed to 18.89 parts of the liquid A.

### Comparative Example 2-2

A thermosensitive recording material (laminate) was produced in the same manner as in Comparative Example 2-1 except that no liquid B was used and 4-propoxy-4′-hydroxydiphenylsulfone as the liquid A was changed to 4-allyloxy-4′-hydroxydiphenylsulfone.

### Comparative Example 2-3

A thermosensitive recording material (laminate) was produced in the same manner as in Example 2-1 except that 4-propoxy-4′-hydroxydiphenylsulfone as the liquid A was changed to 4-allyloxy-4′-hydroxydiphenylsulfone.

### Comparative Example 2-4

A thermosensitive recording material (laminate) was produced in the same manner as in Example 2-2 except that 4-propoxy-4′-hydroxydiphenylsulfone as the liquid A was changed to 4-allyloxy-4′-hydroxydiphenylsulfone.

### Comparative Example 2-5

A thermosensitive recording material (laminate) was produced in the same manner as in Example 2-3 except that 4-propoxy-4′-hydroxydiphenylsulfone as the liquid A was changed to 4-allyloxy-4′-hydroxydiphenylsulfone.

### Comparative Example 2-6

A thermosensitive recording material (laminate) was produced in the same manner as in Example 2-4 except that 4-propoxy-4′-hydroxydiphenylsulfone as the liquid A was changed to 4-allyloxy-4′-hydroxydiphenylsulfone.

### Comparative Example 2-7

A thermosensitive recording material (laminate) was produced in the same manner as in Example 2-5 except that 4-propoxy-4′-hydroxydiphenylsulfone as the liquid A was changed to 4-allyloxy-4′-hydroxydiphenylsulfone.

### Comparative Example 2-8

A thermosensitive recording material (laminate) was produced in the same manner as in Example 2-6 except that 4-propoxy-4′-hydroxydiphenylsulfone as the liquid A was changed to 4-allyloxy-4′-hydroxydiphenylsulfone.

[Evaluation of Thermosensitive Recording Material (Laminate)]

Each of the thermosensitive recording materials (laminates) obtained in Examples and Comparative Examples was evaluated as follows.

<Heat Resistance>

After a thermosensitive recording material where a checkerboard pattern was printed at an applied energy of 0.36 mJ/dot by use of a thermosensitive printer (TH-M2/PS) manufactured by Ohkura Electric Co., Ltd. was left to stand at 60° C. for 24 hours, the print density of a print area was measured with an eXact densitometer manufactured by X-Rite, Incorporated., and the remaining rate (%) was calculated. The results are shown in Table-2. A higher value with respect to the results in the present test indicates more favorable heat resistance.

Remaining rate (%)=[[Print density after test]/[Print density before test]]×100

<Plasticizer Resistance>

After the front and the rear of a thermosensitive recording material where a checkerboard pattern was printed at an applied energy of 0.36 mJ/dot by use of a thermosensitive printer (TH-M2/PS) manufactured by Ohkura Electric Co., Ltd. were wound by DIAWRAP (manufactured by Mitsubishi Chemical Corporation) and left to stand at 20° C. for 2 hours, the print density of a print area was measured with an eXact densitometer manufactured by X-Rite, Incorporated, and the remaining rate (%) was calculated. The results are shown in Table-2. A higher value with respect to the results in the present test indicates more favorable plasticizer resistance.

Remaining rate (%)=[[Print density after test]/[Print density before test]]×100

<Moisture Resistance>

After a thermosensitive recording material where a checkerboard pattern was printed at an applied energy of 0.36 mJ/dot by use of a thermosensitive printer (TH-M2/PS) manufactured by Ohkura Electric Co., Ltd. was left to stand at 40° C. and at a humidity of 90% for 24 hours, the print density of a print area was measured with an eXact densitometer manufactured by X-Rite, Incorporated, and the remaining rate (%) was calculated. The results are shown in Table-2. A higher value with respect to the results in the present test indicates more favorable moisture resistance.

Remaining rate (%)=[[Print density after test]/[Print density before test]]×100

TABLE 2

| | | | | | Heat | Plasticizer | Moisture |
|---|---|---|---|---|---|---|---|
| | | | | | resistance | resistance | resistance |
| | Compound (1) | | Compound (2) | | 60° C.-24 h | 20° C.-2 h | 40° C.-90%-24 h |
| | | Content | | Content | Remaining rate | Remaining rate | Remaining rate |
| | | [% by | | [% by | of image density | of image density | of image density |
| | Compound name | weight] | Compound name | weight] | (%) | (%) | (%) |
| Comparative | 4-Propoxy-4′- | 100 | — | — | 68% | 66% | 73% |
| Example 2-1 | hydroxydiphenylsulfone | | | | | | |
| Example 2-1 | | 97.5 | 4,4′- | 2.5 | 79% | 78% | 87% |
| Example 2-2 | | 85 | Dipropoxydiphenylsulfone | 15 | 92% | 81% | 93% |
| Example 2-3 | | 70 | | 30 | 89% | 78% | 93% |
| Example 2-4 | | 97.5 | 4,4′- | 2.5 | 84% | 83% | 87% |
| Example 2-5 | | 85 | Diallyloxydiphenylsulfone | 15 | 90% | 87% | 93% |
| Example 2-6 | | 70 | | 30 | 86% | 81% | 90% |
| Comparative | 4-Allyloxy-4′- | 100 | — | — | 43% | 51% | 47% |
| Example 2-2 | hydroxydiphenylsulfone | | | | | | |
| Comparative | | 97.5 | 4,4′- | 2.5 | 58% | 60% | 60% |
| Example 2-3 | | | Dipropoxydiphenylsulfone | | | | |
| Comparative | | 85 | | 15 | 75% | 69% | 75% |
| Example 2-4 | | | | | | | |
| Comparative | | 70 | | 30 | 69% | 60% | 69% |
| Example 2-5 | | | | | | | |
| Comparative | | 97.5 | 4,4′- | 2.5 | 54% | 61% | 63% |
| Example 2-6 | | | Diallyloxydiphenylsulfone | | | | |
| Comparative | | 85 | | 15 | 69% | 73% | 76% |
| Example 2-7 | | | | | | | |
| Comparative | | 70 | | 30 | 72% | 67% | 77% |
| Example 2-8 | | | | | | | |

As clear from Table-2, comparison of Comparative Examples 2-3 to 2-5 where 4-allyloxy-4′-hydroxydiphenylsulfone not corresponding to the compound (1) and 4,4′-dipropoxydiphenylsulfone corresponding to the compound (2) were used, with Examples 2-1 to 2-3 where 4-propoxy-4′-hydroxydiphenylsulfone corresponding to the compound (1) and 4,4′-dipropoxydiphenylsulfone corresponding to the compound (2) were used, indicated that Examples 2-1 to 2-3 exhibited excellent results with respect to respective evaluation items such as heat resistance, plasticizer resistance and moisture resistance. Comparison of Comparative Examples 2-6 to 2-8 where 4-allyloxy-4′-hydroxydiphenylsulfone not corresponding to the compound (1) and 4,4′-diallyloxydiphenylsulfone corresponding to the compound (2) were used, with Examples 2-4 to 2-6 where 4-propoxy-4′-hydroxydiphenylsulfone corresponding to the compound (1) and 4,4′-diallyloxydiphenylsulfone corresponding to the compound (2) were used, indicated that Examples 2-4 to 2-6 exhibited excellent results with respect to respective evaluation items such as heat resistance, plasticizer resistance, and moisture resistance.

From the above, the present invention can provide a thermosensitive recording material and a laminate which are favorable in storage characteristics of a print area, such as heat resistance, plasticizer resistance, moisture resistance, oil resistance, and grease resistance.

### INDUSTRIAL APPLICABILITY

The thermosensitive recording material and the laminate of the present invention can be suitably used in any applications such as paper, a film, an IC card, and a friction ballpoint pen.

What is claimed is:

1. A thermosensitive recording material in which a thermosensitive recording layer is provided on a support, wherein the thermosensitive recording layer comprises a compound represented by the following formula (1) and a compound represented by the following formula (2), and a content of the compound represented by formula (2) based on a total amount of these compounds is more than 2.0% by weight and 40% by weight or less: 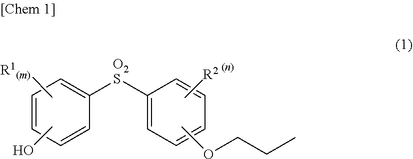
in formula (1), R¹ and R² are optionally the same as or different from each other, and each represents an alkyl group having 1 to 3 carbon atoms, an alkenyl group having 1 to 3 carbon atoms, or a benzyl group, and m and n each independently represent an integer of 0 to 4; 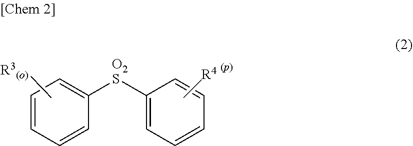
in formula (2), R³ and R⁴ are optionally the same as or different from each other, and each represents an alkyloxy group having 1 to 3 carbon atoms, an alkenyloxy group having 1 to 3 carbon atoms, or a benzyloxy group, and o and p each independently represent an integer of 1 to 3.

2. The thermosensitive recording material according to claim 1, wherein the thermosensitive recording layer comprises 4-propoxy-4′-hydroxydiphenylsulfone as the compound represented by formula (1).

3. The thermosensitive recording material according to claim 1, wherein the thermosensitive recording layer comprises at least one selected from the group consisting of 4,4′-dipropoxydiphenylsulfone, 4,4′-diallyloxydiphenylsulfone, 4,4′-diisopropoxydiphenylsulfone and 4,4′-dibenzyloxydiphenylsulfone, as the compound represented by formula (2).

4. The thermosensitive recording material according to claim 1, wherein the thermosensitive recording layer comprises a leuco dye.

5. The thermosensitive recording material according to claim 4, comprising 10 to 200 parts by weight of the leuco dye based on a total of 100 parts by weight of the compound represented by formula (1) and the compound represented by formula (2).

6. The thermosensitive recording material according to claim 1, wherein the thermosensitive recording layer comprises a sensitizer.

7. The thermosensitive recording material according to claim 6, comprising at least one selected from the group consisting of 1,2-di-(3-methylphenoxy)ethane, 1,2-diphenoxyethane, a fatty acid amide having 10 to 21 carbon atoms, β-benzyloxynaphthalene, diphenylsulfone, p-toluenesulfonamide, and oxalic acid-di-p-methylbenzyl ester, as the sensitizer.

8. A laminate in which a thermosensitive recording layer is provided on a support, wherein the thermosensitive recording layer comprises a compound represented by the following formula (1) and a compound represented by the following formula (2), and a content of the compound represented by formula (2) based on a total amount of these compounds is more than 2.0% by weight and 40% by weight or less: 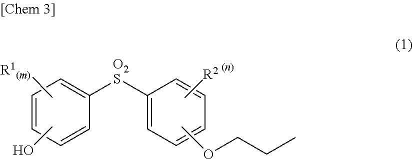
in formula (1), R¹ and R² are optionally the same as or different from each other, and each represents an alkyl group having 1 to 3 carbon atoms, an alkenyl group having 1 to 3 carbon atoms, or a benzyl group, and m and n each independently represent an integer of 0 to 4; 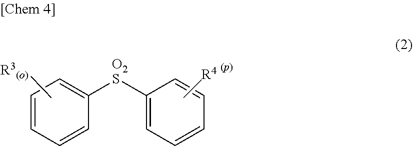
in formula (2), R³ and R⁴ are optionally the same as or different from each other, and each represents an alkyloxy group having 1 to 3 carbon atoms, an alkenyloxy group having 1 to 3 carbon atoms, or a benzyloxy group, and o and p each independently represent an integer of 1 to 3.

9. The laminate according to claim 8, wherein the thermosensitive recording layer comprises 4-propoxy-4′-hydroxydiphenylsulfone as the compound represented by formula (1).

10. The laminate according to claim 8, wherein the thermosensitive recording layer comprises at least one selected from the group consisting of 4,4′-dipropoxydiphenylsulfone, 4,4′-diallyloxydiphenylsulfone, 4,4′-diisopropoxydiphenylsulfone and 4,4′-dibenzyloxydiphenylsulfone, as the compound represented by formula (2).

11. The laminate according to claim 8, wherein the thermosensitive recording layer comprises a leuco dye.

12. The laminate according to claim 11, comprising 10 to 200 parts by weight of the leuco dye based on a total of 100 parts by weight of the compound represented by formula (1) and the compound represented by formula (2).

13. The laminate according to claim 8, wherein the thermosensitive recording layer comprises a sensitizer.

14. The laminate according to claim 13, comprising at least one selected from the group consisting of 1,2-di-(3-methylphenoxy) ethane, 1,2-diphenoxyethane, a fatty acid amide having 10 to 21 carbon atoms, β-benzyloxynaphthalene, diphenylsulfone, p-toluenesulfonamide and oxalic acid-di-p-methylbenzyl ester, as the sensitizer.
